(19) United States
(12) Patent Application Publication
(10) Pub. No.: US 2009/0312826 A1
(43) Pub. Date: Dec. 17, 2009

(54) DEVICE FOR PERIPHERAL INFORMATION INPUT IN TEST SUBJECTS BY MEANS OF ELECTRICAL FIELDS AND CURRENTS

(75) Inventors: Stewart Penny, Maroochydore (AU); Manfred Krauss, Chemnitz (DE); Gerald Grohmann, Jena (DE); Mario Mobius, Leubsdorf (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: HIGH TECH HEALTH INTERNATIONAL LIMITED, Burswood, W.A. (AU)

(21) Appl. No.: 12/300,695

(22) PCT Filed: May 15, 2006
(86) PCT No.: PCT/EP2006/062322

§ 371 Date: Jun. 2, 2009

(51) Int. Cl. A61N 1/04; A61N 1/36

(52) U.S. Cl. 607/149; 607/48

(57) ABSTRACT

The invention relates to a device for peripheral information input in test persons by means of electric fields and currents with at least one electric contacting means for a body part or a test person's extremity, which is arranged releasable with respect to the test person.

The electric contacting means is made of an insulating textile carrier material, the inside of which at least portion-wise exhibiting electrically conductive surfaces, the conductive surfaces, electrically interconnected, either leading to a common outer electric contacting or each are in communication with a separate outer electric contacting. The shape of the textile carrier material is realized corresponding to the anatomy of the test person's relevant body part or relevant extremity and having preferably the shape of a stocking, sock, cuff, headband or glove.

### BACKGROUND

The invention relates to a device for peripheral information input in test persons by means of electric fields and currents according to the preamble of claim 1.

It is known that (minimal) time-variable electric currents may cause stimulating effects when coupled into the cardiovascular system of humans. In this sense, literature talks about information therapy, since the stimulating success may already occur with currents in the range of microamperes. In achieving this set target, the frequency contents of the time-variable stimulation currents play a decisive role [Dertinger, H: Hochwirksame Elektrotherapie gegen Schuppenflechte. Spektrum der Wissenschaft. April 2000; Marino, A A: Modern Bioelectricity. Marcel Dekker New York and Basel 1988; Kruglikov, I L. and H. Dertinger: Stochastic Resonance as a Possible Mechanism of Amplification of Weak Electric Signals in Living Cells. Bioelectromagnetics 15:539-547; Krauss, M: Die natuerlichen elektromagnetischen Signale in unserer Umwelt and deren Simulation als physikalische Therapie. North German Conference of Complementary Medicine, 22-23 Jun., 2002 Wilhelmshaven/Germany].

As everybody knows, the veins in the cardiovascular system have the function of returning the blood to the heart and to store the blood volume that is not required for the circulation in process. The respiration thereby acts like a pressure-suction pump [Hach, W et al.: Phlebographie der Bein-und Beckenvenen. Schnetztor Konstanz 1996] so that an increase of the venous blood reflux is possible by an external electric peripheral muscle stimulation in the rhythm of the respiration. As soon as the calf muscle in the lower leg is tensed, it is known that the lumen of the conducting veins situated behind is restricted to a narrow gap by the swelling muscle bellies of the calf muscles. With a relaxation of the musculature, the venous vessel system immediately reassumes a normal width. The same is valid for other muscle areas of the circulatory system [Hach, W et al.: Phlebographie der Bein-und Beckenvenen. Schnetztor Konstanz 1996].

Without taking these essential physiological contexts as a basis, numerous micro-devices have been developed especially for an electric muscle and nerve stimulation, as shown, for example, in the U.S. Pat. No. 6,615,080.

As far as the prior art is concerned, reference should be made to the studies of Hogan [Hogan M C, Grassi B, Samaja M, Stary C M, Gladden L B: Effect of concentration frequency on the contractile and noncontractile phases of muscle venous blood flow. J Appl Phys 2003; 95: 1139-44] and Lyons [Lyons G M, Leane G E, Clarke-Moloney M, O'Brien J V, Grace P A: An investigation of the effect of electrode size and electrode location on comfort during stimulation of the gastrocnemius muscle. Med Eng Phys 2004; 26: 873-8]. There is reported based on practical test results on rabbits that for an optimum increase of the venous blood reflux the gastrocnemius muscle pump has to be activated in a rhythm of 0.25 to 0.5 contractions/s (≡Hz) by means of electric stimulation. In this case, the venous blood flow increases with an increasing contraction frequency.

According to the prior art, the (micro)currents needed for a stimulation mostly are coupled in via tenselectrodes. The disadvantage of tenselectrodes mainly is the limited service life caused by the adhesion at the input site. Therefore, applicators have been developed which in part wrap around the foot.

In summary, it has hence to be noted that the known peripheral applicators often do not satisfy the medical circumstances and regularities especially of a peripheral arterial stimulation of the circulatory system. Thus, it is known from the physiology of the cutaneous blood vessel system [Altmeyer, P et al: Kutane Mikrozirkulation. Springer Berlin Heidelberg 1997, ISBN 3-540-62564-X Gb] that the control of the cutaneous blood flow in the acral and distal parts of the extremities such as feet and hands is influenced by sympathetic fibers. According to this, these acral regions exhibit the highest values and the largest variabilities in blood flow. Since, moreover, the nerve fibers, besides the blood vessel system, represent well conducting media of the human body, the necessary information for stimulating the arterial circulatory system should be coupled into these peripheral regions.

### SUMMARY

From the afore-mentioned, it is therefore an object of the invention to propose an improved device for peripheral information input in test person by means of electric fields and currents, which aids in avoiding pain sensations at a low pain tolerance even with an increased stimulation amplitude, and wherein sensations of discomfort are not imposed on the test person when carrying the device.

The solution of the object ensues with the feature combination according to claim 1, with the depending claims representing at least suitable configurations and further developments.

According to the invention, sensitive areas in the region of sensible nerve fibres are left free. According to the invention, the application of voltages takes place between two determined peripheral regions so that a corresponding current flow will occur.

As examples for the information input into the circulatory system should be mentioned:
Foot sole up to the acral region of the left and right feet. Here, a current flow occurs from this peripheral region across all of the foot, the lower and upper leg of the one leg including musculature, back to the upper and lower leg as well as to the periphery of the other leg including musculature. At the same time, an information forwarding takes place to the central nervous system consisting of spinal cord and brain. A parallel input between both of the peripheral regions and the cervical vertebrae may likewise take place.
Acral finger regions, hand, lower and upper arm on the left side including musculature, across upper and lower arm, as well as hand and acral finger regions on the right side including musculature. In analogy to the legs, a parallel input may likewise be effected between the arms and the cervical vertebrae.
Input at two or four extremities at the same time, in the sense of a two cell or four cell bath.
Finger or foot region to characteristic locations of the spinal cord.
Between an amputation stump and the other part of the periphery or to the spinal cord.

According to the invention, an external peripheral electric muscle stimulation in the rhythm of an average respiration frequency of about 0.15 . . . 0.23 . . . 0.3 Hz including further frequencies allows the venous blood reflux to be increased and hence a possible thrombosis and pulmonary embolism to be prevented. As advantageous examples for such an information input indirectly into the venous circulatory system should be mentioned:
Beginning and end of the muscle belly of the calf muscle at the lower leg,
Beginning and end of the muscle bellies of the upper leg muscles as may be necessary inter alia in case of an amputation of the lower leg,
Beginning and end of the muscle bellies at the lower and upper arms.

The device for peripheral information input in test persons by means of electric fields and currents is based on at least one electric contacting means for a body part or a test person's extremity, which is arranged so as to be releasable with respect to the test person.

Specifically, the electric contacting means is made of an insulating textile carrier, the inner side of which at least portion-wise exhibiting electrically conductive surfaces, the conductive surfaces, electrically interconnected, either leading to a common outer electric contacting or each are in communication with a separate outer electric contacting.

The shape of the textile carrier material corresponds to the anatomy of the relevant body part or the relevant extremity and is preferably realized as a stocking, sock, cuff, headband or glove or in a similar way.

According to the invention, the conductive surfaces may exhibit moisturizing or moisture-storing filaments so that the contact resistance between the device inner side and the test person's skin may be minimized.

The conductive surfaces may be incorporated into the textile material by non-weaving, knitting or weaving of conductive fibers.

As a complement, the textile carrier material exhibits elastic properties so as to bring about the desired good surface contact adhesion to the extremity. Elastic threads or yarns may be woven or knit into the textile material for this purpose.

The external electric contacting preferably is realized as a releasable connection in the form of a conductive press or Velcro contact.

When forming the electric contacting means as a stocking or a sock, the inner side of the foot sole extending to the dorsal toe region and inclosing the same at least in part is realized as a conductive surface.

This electrically conductive surface is expanded by a narrow conductive strip extending into the calf part of the stocking.

The outer electric contacting may then be arranged at the free end of the narrow conductive strip introduced into the calf part.

In a variation of this configuration, the narrow conductive strip leads into the calf part of the stocking across a heel portion.

When forming the electric contacting means as a stocking or sock, conductive surfaces may be arranged in the calf part. These conductive surfaces preferably are formed as ring-shaped, completely surrounding strips or strip portions. In this case, an outer electric contacting is situated at each strip or each strip portion.

When forming the electric contacting means as a glove, the conductive surface covers the region of all of the fingers, i.e. there exists an electric connection between these portions covering the fingers.

### BRIEF DESCRIPTION OF THE DRAWINGS

The invention will be explained in more detail by means of exemplary embodiments and figures.

These show:

FIG. 1 shows foot-sock applicators,

FIG. 2 shows calf muscle applicators,

FIG. 3 shows stocking-stocking applicators with a full plating, and

FIG. 4 shows a glove applicator.

### DETAILED DESCRIPTION OF THE PREFERRED EMBODIMENTS

The realization examples follow the basic idea of how by means of a constructive adaptation to the discussed physiological circumstances an optimum peripheral information input may be realized by means of electric fields and currents.

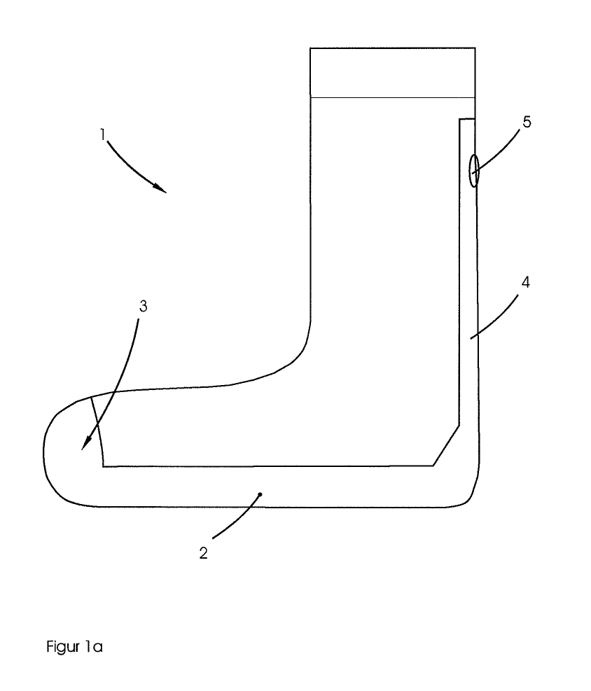

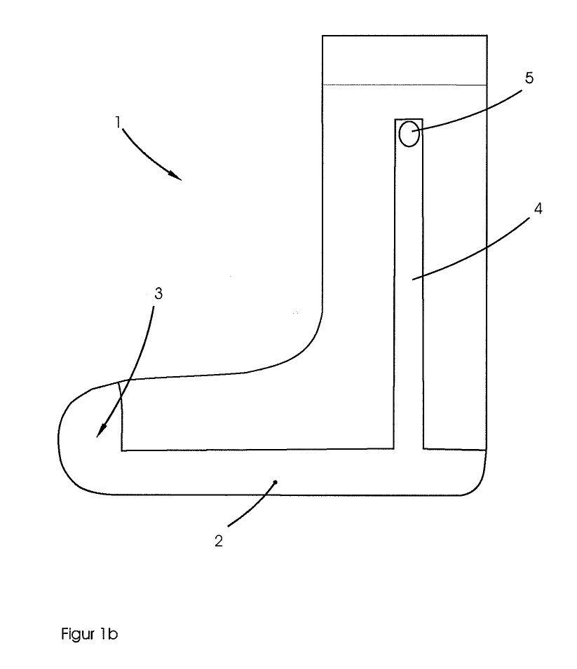

In FIG. 1a, 1b illustrate textile foot-sock applicators 1, with the information input being possible on the inside via an expanded foot sole 2 well into the dorsal toe region. The general case that the inside of the applicator is fully plated, here should be included. By means of known technologies such as knitting, non-weaving, raschel-knitting, weaving or non-woven methods, electrically conductive silver fibers/surfaces 3 are incorporated into the sole region during the production.

The information supply to the electric fibers/surfaces in FIG. 1 is realized through a narrow extension of the electrode surfaces 4 well up to a variable releasable plug connection 5 in the upper region.

If the general case is present that the inside of the foot-sock applicator is fully plated, the variable releasable plug connection is likewise attached to the upper sock region. From this plug connection, a connector cable up to the central control device is an integral part of the invention. A stocking in each case then acts as an electrode. For the pair-wise application of the “left and right foot” applicator, one control device is required. The outer surface is configured like a normal sock. A parallel operation of both of the foot applicators may boost the stimulation effect. In such a case, the counterelectrode has to be applied to the spinal cord, preferably in the region of the lumbar vertebrae (not shown).

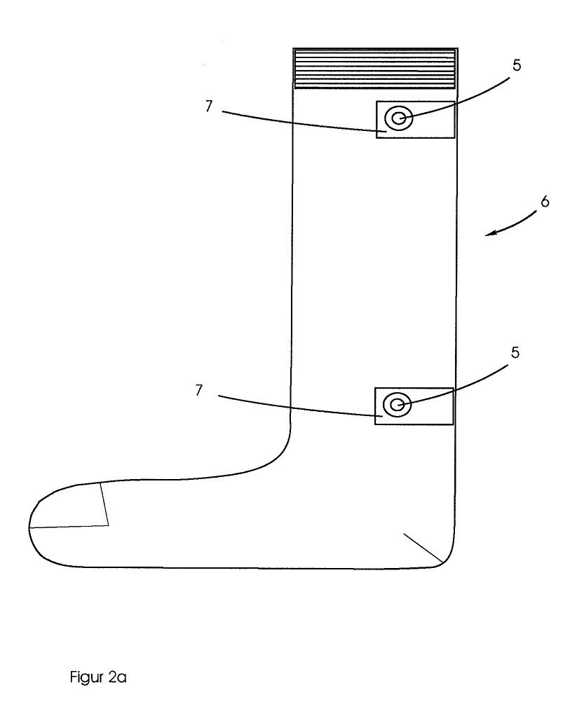

From FIG. 2a, 2b possibilities for constructing a calf muscle applicator 6 can be seen, where an information input takes place at the beginning and the end of a muscle belly 7. Here, the special case should be included that this calf muscle applicator merely consists of two conductive tapes and these are worn around the lower leg, especially around the beginning and the end of the muscle belly. The same is true for the upper leg and the entire arm region.

FIG. 2a shows a realization example of a calf muscle applicator 6 having discontinuous, i.e. not surrounding conductive strips 7, whereas FIG. 2b shows an embodiment having completely surrounding conductive strips 7.

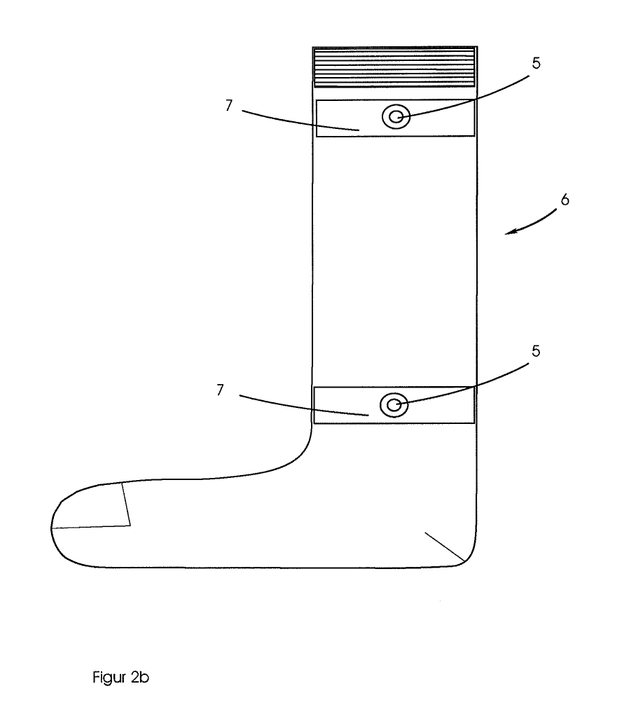

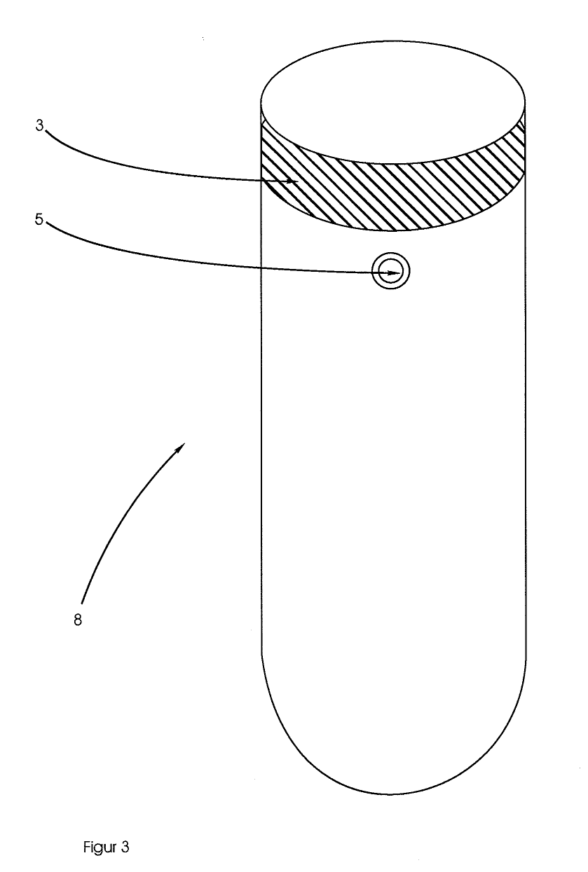

FIG. 3 shows a basic configuration of a stocking-stocking applicator 8, where an information input takes place in analogy to the foot-sock applicator, and electrically conductive silver fibers/surfaces 3 are likewise incorporated into the inside of the applicator at a full plating. The integration of two conductive tapes fur muscle activation is likewise possible.

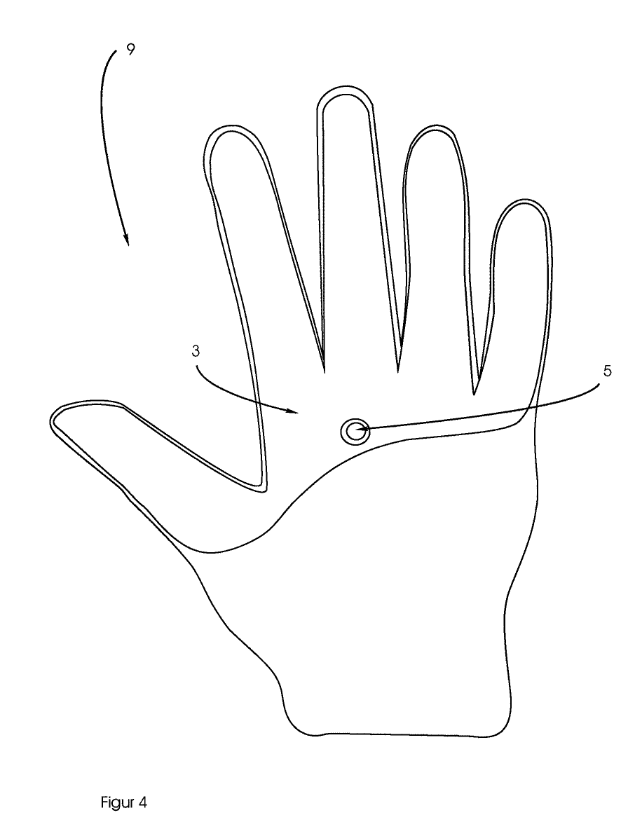

A peripheral glove applicator 9 is illustrated in FIG. 4, wherein an information input especially into the arterial circulatory system takes place across the entire finger region. In this case, the fingers are completely surrounded by the electrically conductive material 3 as being the inner component of the applicator, which is introduced during the production in analogy to FIG. 1. Preferably, electrically conductive silver fibers/surfaces are also used in analogy to the foot-sock applicator. An electric connection between the fingers ensures a uniform information supply. The information supply to the surface is ensured, in analogy to FIG. 1, through a variable releasable plug connection 5.

One glove in each case acts as an electrode so that for the pair-wise application of the “left and right hand” glove applicator one control device is required in analogy to the foot-sock applicator. The outer surface is shaped like a normal glove. A parallel stimulation through both of the applicators is likewise intended such that the counterelectrode in each case is applied in the region of the spinal cord, preferably to the lumbar vertebrae.

The contact between the human body and the described applicators according to FIGS. 1 to 4, due to their tightly fitting construction, is nearly free of losses, the contact resistance is low. The electrically active surfaces are each integrated on the inner side. Thereby the described properties of silver are likewise utilized according to the invention, hence a low tendency of corrosion and the medically important effect of combating bacterial and fungal infection associated with the possibility of an information input into the cardiovascular system (arterial and venous blood vessel system, autonomic nervous system).

For the purpose of further reducing the contact resistance between the electrically active surfaces of the applicators and the skin, and hence also the pain sensation at a relatively low electric stimulation, a moisturization of the skin in the general sense is performed (e.g. with gel used in ultrasonic examinations, moisturization with water, moisturizing filaments on the inside of the applicator, or foot cream).

Possible medical application fields of the invention are seen inter alia in: prevention of arterial and venous thrombosis, diabetes (diabetic foot), peripheral circulatory disturbances, wound healing processes, high blood pressure, depressions (due to systemic action via vegetative fibers), Restless Legs Syndrome, Raynaud Syndrome, and other functional circulatory disturbances. Corresponding frequency stimulation programs are always required.

### List of Reference Numerals

1 foot-sock applicator
2 foot sole
3 conductive inner surface
4 extension of electrode surface
5 plug connection/outer contact
6 calf muscle applicator
7 information input through conductive strips
8 stocking-stocking applicator
9 glove applicator

1. Device for peripheral information input in test persons using electric fields and currents comprising at least one electric contact for a body part or a test person's extremity, which is arranged releasable with respect to the test person, the electric contact is made of an insulating textile carrier material, an inside of which at least portion-wise includes electrically conductive surfaces, the conductive surfaces are electrically interconnected, and lead to a common outer electric contacting or each are in communication with a separate outer electric contacting, and a shape of the textile carrier material corresponds to the anatomy of the relevant body part or the relevant extremity and is formed as a stocking, sock, cuff, headband or glove.

2. Device in accordance with claim 1, wherein the conductive surfaces exhibit moisturizing or moisture-storing filaments.

3. Device in accordance with claim 1, wherein the conductive surfaces are incorporated into the textile material by non-weaving, knitting or weaving of conductive fibers.

4. Device in accordance with claim 1, wherein the textile material comprises elastic threads or yarns for obtaining a predetermined press fit of the device.

5. Device in accordance with claim 1, wherein the outer contacting exhibits a releasable connection in the form of a conductive press or Velcro contact.

6. Device in accordance with claim 1, wherein when forming the electric contact as a stocking or sock, an inner side of the foot sole is formed as a conductive surface up to a dorsal toe region.

7. Device in accordance with claim 6, wherein the electrically conductive surface is expanded by a narrow conductive strip extending into the calf part of the stocking.

8. Device in accordance with claim 7, wherein the outer contacting is arranged at a free end of the narrow conductive strip introduced into the calf part.

9. Device in accordance with claim 7, wherein the narrow conductive strip leads into the calf part of the stocking across a heel portion.

10. Device in accordance with claim 1, wherein when forming the electric contact as a stocking or sock, conductive surfaces are contained in the calf part.

11. Device in accordance with claim 10, wherein the conductive surfaces are formed as ring-shaped, completely surrounding strips or strip portions, and an outer contacting is situated at each strip or each strip portion.

12. Device in accordance with claim 1, wherein when forming the electric contact as a finger glove, the conductive surface covers a region of all of the fingers.

13. Device in accordance with claim 1, further comprising a voltage source adapted to apply a respiration-dominant stimulation voltage to the conductive surfaces, with a frequency of the applied voltage being in a range of 0.15 Hz to 3 Hz.

14. Device in accordance with claim 13, wherein an average of the frequency of the applied voltage is 0.23 Hz.
